(19) United States
(12) Patent Application Publication
(10) Pub. No.: US 2022/0362611 A1
(43) Pub. Date: Nov. 17, 2022

(54) Handheld Exercise Device

(71) Applicant: John Michael Thomas, Wayne, NJ (US)

(72) Inventor: John Michael Thomas, Wayne, NJ (US)

(21) Appl. No.: 17/744,286

(22) Filed: May 13, 2022

Related U.S. Application Data
(60) Provisional application No. 63/188,065, filed on May 13, 2021.

(51) Int. Cl. A63B 21/00; A63B 21/06; A63B 24/00; A63B 71/06

(52) U.S. Cl. CPC A63B 21/0004; A63B 21/0603; A63B 21/0608; A63B 24/0062; A63B 71/0619; A63B 21/4035; A63B 24/0087; A63B 2225/74; A63B 2225/54; A63B 2220/833; A63B 2220/36; A63B 2220/35

(57) ABSTRACT

A handheld exercise device is an apparatus that enables the user to exercise the upper body due to the load applied on the user's arms from the centripetal force created by moving the apparatus using one or two hands. To do so, the apparatus may include an O-ring housing, a crossbar, a bearing assembly, and a control system. The O-ring housing is designed to retain the bearing assembly in such a way that the user can move the bearing assembly in a circular motion within the O-ring housing. The crossbar facilitates the handling of the O-ring housing with one or two hands in order for the user to move the bearing assembly in a circular motion within the O-ring housing. The control system helps the user keep track of the movement of the bearing assembly so that the user can measure the exercise progress while handling the apparatus.

### FIELD OF THE INVENTION

The present invention generally relates to exercise equipment. More specifically, the present invention provides a handheld exercise device equipped with state-of-the-art technology to provide improved physical strength and conditioning, mental health, and body balance.

### BACKGROUND OF THE INVENTION

Exercise equipment comes in various sizes and configurations designed to target different areas of the body. Exercise equipment is often designed to assist in the performance of a variety of exercises to promote increased physical health and mental wellbeing. There are various handheld exercise devices that are designed to help exercise the upper body of the user. For example, different dumbbells are available that help users exercise their arms and shoulders by performing different movements and actions. However, few handheld exercise devices are designed to utilize centripetal force resulting from the rotational motion of the exercise device to promote the exercising of the upper body. Most of the current exercise devices available require linear motion to perform the exercise activities, which limit the effectiveness of the exercise. So, there is a need for a handheld exercise device that utilizes centripetal force to promote a more comprehensive exercise that targets the upper body of the user.

An objective of the present invention is to provide a handheld exercise device designed to utilize the centripetal force resulting from the rotational motion of the device to exercise the upper body of the user. The present invention is designed to make the user rotate portions of the device to cause centripetal force that makes the user tighten the grip on the device to maintain smooth control. Another objective of the present invention is to provide a handheld exercise device that provides various stimuli that engages the user while exercising. Additional benefits and features of the present invention are further discussed in the sections below.

### SUMMARY OF THE INVENTION

The present invention is a handheld exercise device that provides a more practical, effective, and technologically advanced alternative to present day handheld exercise equipment. The present invention, also known as The Corbit Challenge, promotes muscle toning, core strengthening, cardiovascular health, and a significantly higher level of body balance for the user. Further, the present invention stimulates the mind and body while exercising. The present invention is preferably a ring-shaped structure housing several bearings that can move freely within the ring-shaped structure. In addition, the present invention includes a grip portion that enables the user to easily handle the ring-shaped structure.

The present invention works by grasping the device with two hands, with palms up or down. Then, by moving the device in a specific direction, the ball bearings move in a circular motion within the ring-shaped structure. The circular motion of the ball bearings within the ring-shaped structure results on a centripetal force acting on the arms of the user. As the user increases the speed of the bearings, while maintaining a smooth rhythmic motion, the tension on the user's arms increases, causing the user to tighten their grip to maintain smooth control. This action facilitates multiple arm, shoulder, chest, and core muscles to engage. Further, when the user reverses the direction of motion of the bearings and/or switches hand grip position, a different set of muscle groups will engage. The present invention can also be held with one hand, and in doing so, increases the level of muscle fatigue. Furthermore, the present invention may include various stimulating features that engages the user while exercising, such as lights, sounds, etc. Additional features include means to track the exercise progress and other health-related parameters.

### BRIEF DESCRIPTION OF THE DRAWINGS

FIG. 1 is a top front perspective view showing the present invention.

FIG. 2 is a bottom rear perspective view showing the present invention.

FIG. 3 is a front view showing the present invention.

FIG. 4 is a horizontal cross-sectional view of the present invention taken along line 4-4 in FIG. 3.

FIG. 5 is a vertical cross-sectional view of the present invention taken along line 4-4 in FIG. 3.

FIG. 6 is a top front perspective view showing the bearing assembly of the present invention.

FIG. 7 is a side view showing the bearing assembly of the present invention.

FIG. 8 is a vertical cross-sectional view of the bearing assembly of the present invention taken along line 8-8 in FIG. 7.

FIG. 9 is a front view showing the present invention, wherein the bearing assembly is shown moved within the hollow ring body in a clockwise direction.

FIG. 10 is a schematic showing the electrical connections and the electronic connections of the present invention, wherein the electrical connections are shown in dashed lines, and wherein the electronic connections in dotted lines.

### DETAIL DESCRIPTIONS OF THE INVENTION

All illustrations of the drawings are for the purpose of describing selected versions of the present invention and are not intended to limit the scope of the present invention.

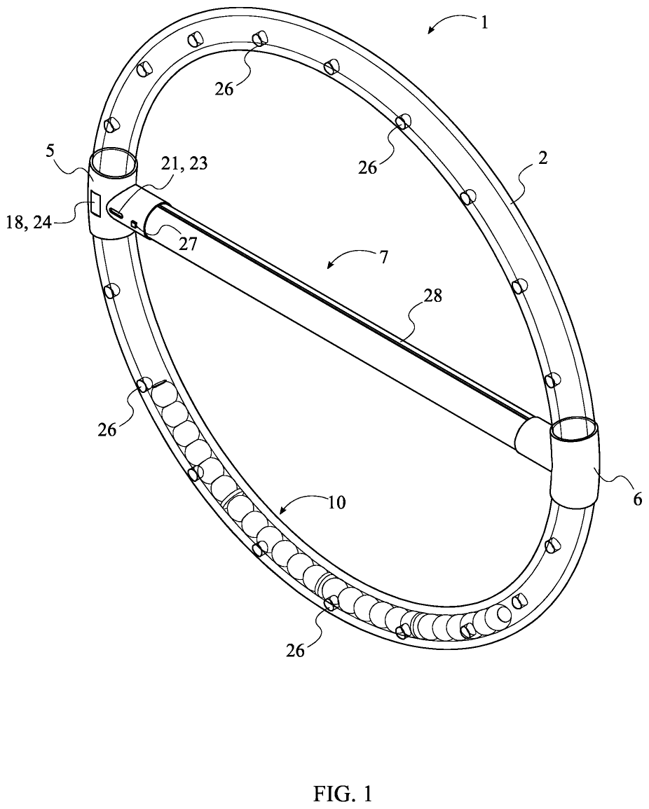

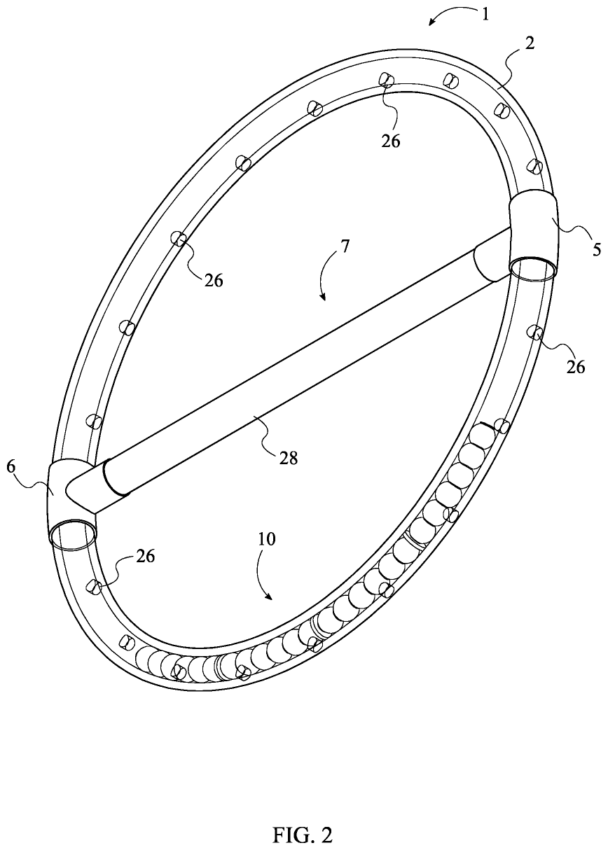

The present invention is a handheld exercise device that enables users to exercise the upper body due to stress on the user's arms from the centripetal force created by moving the present invention using one or two hands. As can be seen in FIG. 1 through 3, to do so, the present invention may comprise an O-ring housing 1, a crossbar 7, a bearing assembly 10, and a control system 18. The O-ring housing 1 retains the bearing assembly 10 in such a way that the user can move the bearing assembly 10 in a circular motion while handling the O-ring housing 1. The crossbar 7 facilitates the handling of the O-ring housing 1 with one or two hands in order for the user to move the bearing assembly 10 in a circular motion within the O-ring housing 1. The control system 18 helps the user keep track of the movement of the bearing assembly 10 so that the user can measure the exercise progress while handling the O-ring housing 1.

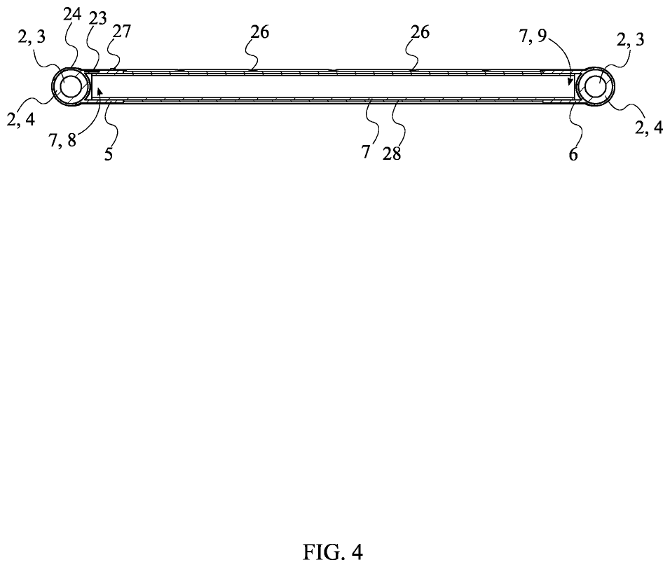

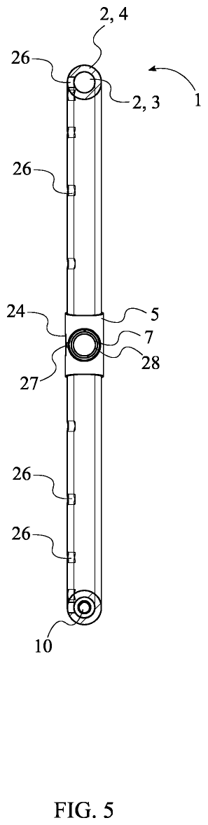

The general configuration of the aforementioned components enables the user to efficiently exercise the upper body while remaining engaged during the exercise. As can be seen in FIG. 1 through 5, the O-ring housing 1 is designed to retain the bearing assembly 10 while enabling the movement of the bearing assembly 10 within the O-ring housing 1. To do so, the O-ring housing 1 comprises a hollow ring body 2, a first coupler 5, and a second coupler 6. The hollow ring body 2 is designed as a round hollow structure with a cross-section preferably including a center hollow space 3 and a thin outer wall 4. The inner diameter of the thin outer wall 4 is preferably large enough to accommodate the bearing assembly 10 as well as to obstruct with the movement of the bearing assembly 10. Further, the thickness of the thin outer wall 4 is thick enough to sustain the load from the movement of the bearing assembly 10. The first coupler 5 and the second coupler 6 facilitate the mounting of the crossbar 7 to the hollow ring body 2. In a first embodiment, the hollow ring body 2 may include an inner ring that may be made from 1-inch (in.) PEX tubing and may embody an assortment of colors, diameters, and weight classes. In the first embodiment, the hollow ring body 2 is a 1⅝ in. clear flexible tubing which slides over the 1 in. PEX tubing and serves as a sound suppressor.

As can be seen in FIG. 1 through 5, the crossbar 7 is an elongated cylindrical structure with a length matching the inner diameter of the hollow ring body 2. The outer diameter of the crossbar 7 is large enough to match the outer diameter of the thin outer wall 4 of the hollow ring body 2. Due to the cylindrical elongated design of the crossbar 7, the crossbar 7 comprises a first crossbar end 8 and a second crossbar end 9 corresponding to the terminal ends of the crossbar 7. Further, the control system 18 is equipped into the present invention to provide the means for the user to keep track of the exercise progress. To do so, the control system 18 comprises a controller 19, a tachometer 20, and a power source 21. The tachometer 20 enables the user to keep track of how many times the bearing assembly 10 has gone around the hollow ring body 2. The controller 19 processes the electrical signals received from the tachometer 20, while the power source 21 provides the current necessary for the operation of the tachometer 20 and the controller 19. In the first embodiment, the crossbar 7 is a ¾ in. PVC and/or plastic cylindrical tube that also acts as a conduit and compartment for the wiring, the power source 21, and other electrical components.

As can be seen in FIG. 1 through 5, to assemble the present invention, the first coupler 5 is positioned opposite to the second coupler 6 across the hollow ring body 2. The positioning of the first coupler 5 and the second coupler 6 are arranged to match the first crossbar end 8 and the second crossbar end 9. This results in the crossbar 7 being positioned along the diameter of the hollow ring body 2. To secure the parts together, the first crossbar end 8 is connected to the hollow ring body 2 by the first coupler 5. Similarly, the second crossbar end 9 is connected to the hollow ring body 2 by the second coupler 6. This way, the crossbar 7 is secured to the hollow ring body 2 in a fixed position so that the user can control the movement of the hollow ring body 2 via the crossbar 7. To protect the electrical and electronic components of the control system 18 from the surroundings, the controller 19 and the power source 21 are mounted within the crossbar 7. On the other hand, the bearing assembly 10 is slidably mounted within the hollow ring body 2 so that the bearing assembly 10 does not get into the crossbar 7. Further, the tachometer 20 is electronically connected to the controller 19 so that the signals generated by the tachometer 20 are processed by the controller 19. In addition, the tachometer 20 and the controller 19 are electrically connected to the power source 21 so that the power source 21 transmits the necessary current to the tachometer 20 and the controller 19. Furthermore, the tachometer 20 is operatively coupled to the bearing assembly 10 to track the movement of the bearing assembly 10 within the hollow ring body 2. The tachometer 20 is preferably used to measure the number of revolutions the bearing assembly 10 has completed along the hollow ring body 2 in a period of time. For example, the tachometer 20 can be designed to measure the Revolutions per Minute (RPM) of the bearing assembly 10 moving within the hollow ring body 2. In other embodiments, the tachometer 20 can be arranged to measure the velocity of the bearing assembly 10 in different units.

As can be seen in FIG. 1 through 5, to ensure the engagement of the user while using the present invention, the hollow ring body 2 is preferably made from a translucent plastic material. This way, the user can visually see the movement of the bearing assembly 10 within the hollow ring body 2 while exercising. The translucent plastic material can be made from any translucent material including, but not limited to, clear PEX tubing. Unlike the hollow ring body 2, the crossbar 7, the first coupler 5, and/or the second coupler 6 may be made from non-translucent material. Further, the present invention can be made in a variety of weight classes, materials, and sizes. For larger users, an O-ring housing 1 of larger diameter can be provided with a corresponding larger crossbar 7. For smaller users, an O-ring of smaller diameter with a corresponding smaller crossbar 7 can be provided.

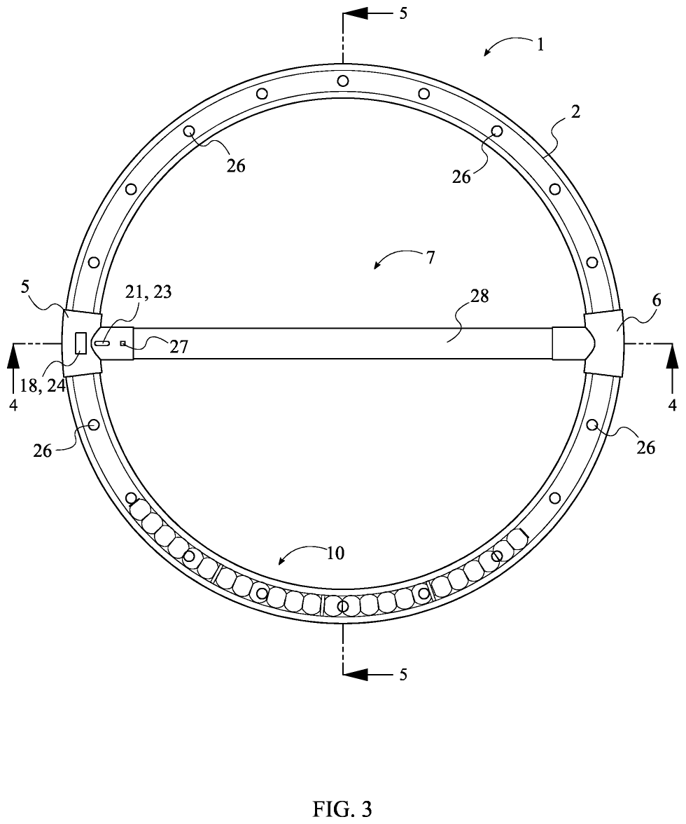

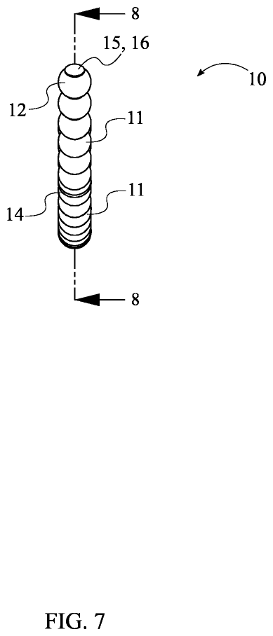

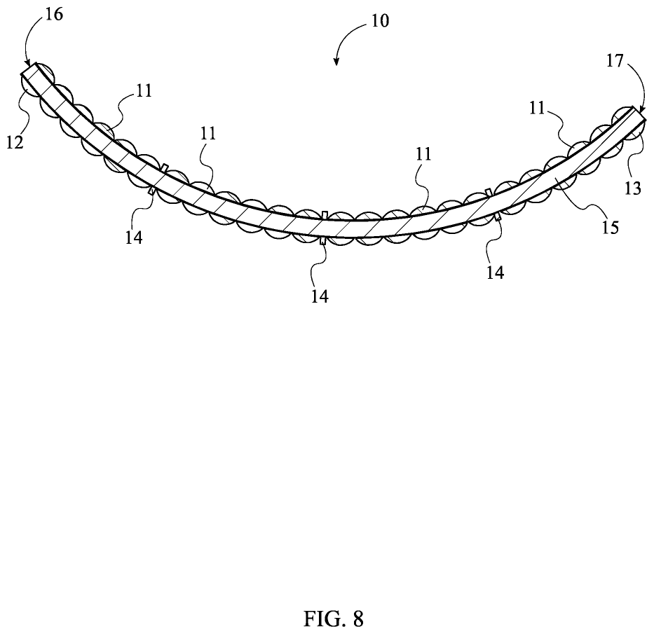

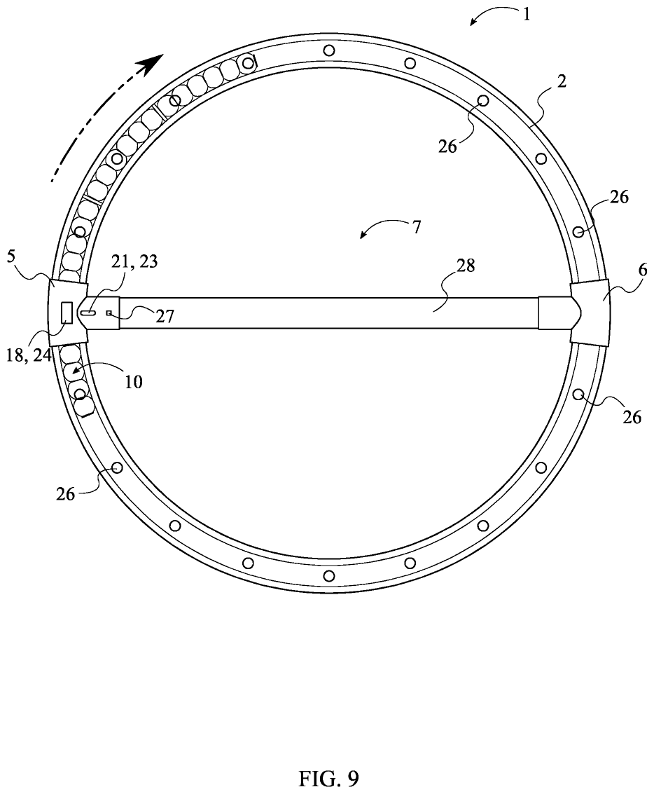

As can be seen in FIG. 3 through 9, the bearing assembly 10 is designed to freely move within the hollow ring body 2 with the least friction. In addition, the bearing assembly 10 is preferably designed to move as a single body to increase the stress on the user's arms. To do so, the bearing assembly 10 may comprise a plurality of bearings 11 and a plurality of magnets 14. The plurality of bearings 11 preferably includes several perforated ball bearings with a diameter matching the inner diameter of the thin outer wall 4 of the hollow ring body 2. The plurality of magnets 14 preferably includes enough ring magnets with a diameter matching the diameter of the plurality of bearings 11. The plurality of magnets 14 is included to engage with the tachometer 20 to help with the tracking of the movement of the bearing assembly 10. Accordingly, the tachometer 20 is a magnetic tachometer 20 that reacts to the movement of the plurality of magnets 14, which move along the plurality of bearings 11. To ensure that the tachometer 20 tracks the movement of the bearing assembly 10, the plurality of magnets 14 is interspersed among the plurality of bearings 11 within the hollow ring body 2. The plurality of magnets 14 can include one or more magnets evenly distributed among the plurality of bearings 11. Further, the tachometer 20 is positioned adjacent to the first coupler 5 and is mounted within the hollow ring body 2. This way, as the plurality of bearings 11 and the plurality of magnets 14 move adjacent to the first coupler 5 within the hollow ring body 2, the tachometer 20 sends signals to the controller 19 which are then processed to determine the completed revolutions along the hollow ring body 2. In a first embodiment, the plurality of bearings 11 includes thirty % in. drilled-through steel ball bearings and the plurality of magnets 14 includes three ½ in. by ¼ in. by ¼ in neodymium rare earth ring donut magnets N42. Further, the bearing assembly 10 helps control the overall weight of the present invention. For example, the overall weight can be controlled by modifying the number of bearings and/or magnets. In addition, the overall weight can be controlled by utilizing bearings made from heavier/lighter materials. The control of the overall weight enables the present invention to be catered to different user strengths. For example, a heavier embodiment can be provided for larger users, while a lighter embodiment can be provided for smaller users. In alternate embodiments, different means of controlling the overall weight of the present invention can be utilized.

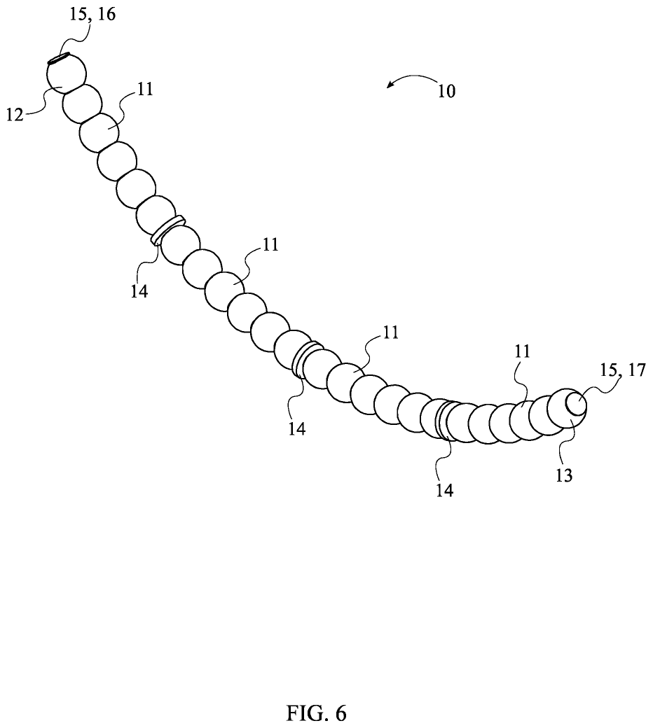

As can be seen in FIG. 6 through 8, to ensure that the plurality of bearings 11 and the plurality of magnets 14 move together, the bearing assembly 10 may further comprise a wire retainer 15. The wire retainer 15 is long enough to maintain the plurality of bearings 11 and the plurality of magnets 14 together as the bearing assembly 10 moves within the hollow ring body 2. The wire retainer 15 comprises a first wire end 16 and a second wire end 17 corresponding to the terminal ends of the wire retainer 15. The plurality of bearings 11 comprises a first terminal bearing 12 and a second terminal bearing 13 corresponding to the bearings positioned at the ends of the bearing assembly 10. The plurality of bearings 11 and the plurality of magnets 14 are distributed along the wire retainer 15. In addition, the wire retainer 15 traverses through each of the plurality of bearings 11 and the plurality of magnets 14. This way, the wire retainer 15 passes through the whole bearing assembly 10 without obstructing the movement of the bearing assembly 10 within the hollow ring body 2. Further, the first wire end 16 is connected to the first terminal bearing 12 while the second wire end 17 is connected to the second terminal bearing 13. This ensures that the plurality of bearings 11 and the plurality of magnets 14 are kept together while moving inside the hollow ring body 2. In the first embodiment, the bearing assembly 10 is held together via a 19-gauge galvanized steel wire. Further, the bearing assembly 10 is permanently sealed and lubricated for smooth low-friction ball bearing roll.

As can be seen in FIGS. 1 through 3 and 10, to help the user track the RPM of the bearing assembly 10, the control system 18 may further comprise a display 24. The display 24 is preferably a small digital display 24 that shows the RPM of the bearing assembly 10 as the user exercises. To do so, the display 24 is externally integrated onto the first coupler 5 so that the display 24 is easily accessible to the user while handling the crossbar 7. Further, the display 24 is electronically connected to the controller 19 to receive the signals from the controller 19 corresponding to the measured RPM from the tachometer 20. The display 24 is also electrically connected to the power source 21 to receive the current necessary for the operation of the display 24. In other embodiments, the display 24 can be designed to show other data from different sensors equipped within the hollow ring body 2.

As can be seen in FIGS. 1 through 3 and 10, to increase the engagement of the user while exercising, the present invention may further comprise a plurality of magnetically activated lights 26. Each of the plurality of magnetically activated lights 26 is designed to turn on as the plurality of magnets 14 along with the plurality of bearings 11 move adjacent to the plurality of magnetically activated lights 26. To do so, each of the plurality of magnetically activated lights 26 includes a light source and a magnetic switch. The plurality of magnetically activated lights 26 is also radially distributed along the hollow ring body 2 so that the movement of the bearing assembly 10 is visually highlighted by the plurality of magnetically activated lights 26. Further, each of the plurality of magnetically activated lights 26 is externally integrated onto the hollow ring body 2. The light source of each of the plurality of magnetically activated lights 26 is preferably positioned adjacent to the outer surface of the hollow ring body 2, while the magnetic switch is positioned adjacent to the inside of the hollow ring body 2. The plurality of magnetically activated lights 26 is also electrically connected to the power source 21 to receive the current necessary for the operation of each magnetically activated light. This way, as the plurality of magnets 14 moves along with the rest of the bearing assembly 10 within the hollow ring body 2, the nearest magnetically activated lights are powered on. As the bearing assembly 10 moves away, the magnetically activated lights are powered off. In other embodiments, different light sources can be utilized to engage the user while exercising.

As can be seen in FIGS. 1 through 3 and 10, in some embodiments, the user may have the choice of deactivating the plurality of magnetically activated lights 26 if the user feels too distracted by the magnetically activated lights. To do so, the present invention may further comprise a power switch 27 that enables the user to manually activate/deactivate the plurality of magnetically activated lights 26. The power switch 27 is externally integrated onto the first coupler 5, adjacent to the display 24, so that the power switch 27 does not obstruct with the grip of the user on the crossbar 7. Further, the plurality of magnetically activated lights 26 is electrically connected to the power source 21 by the power switch 27 so that the user can manually control the current flow to the plurality of magnetically activated lights 26. Thus, the user can choose if the plurality of magnetically activated lights 26 is activated while exercising.

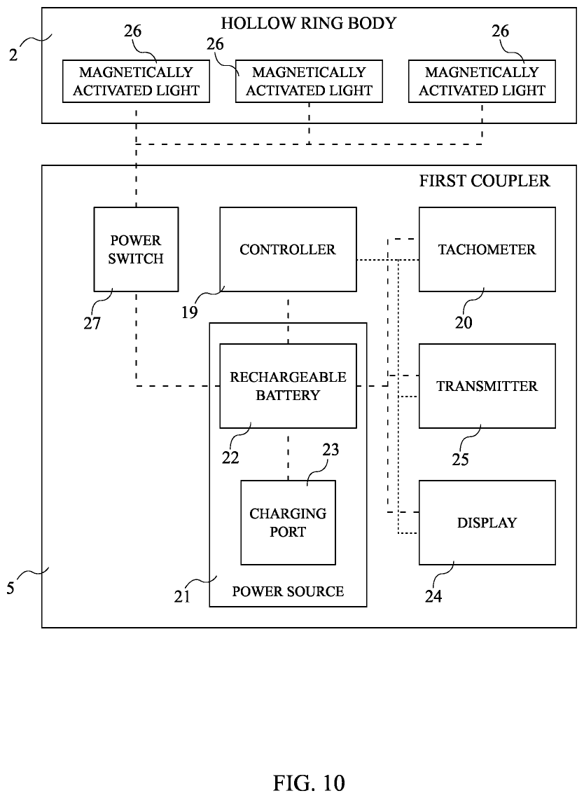

As can be seen in FIG. 10, to present invention preferably provides means to ensure that the control system 18 always has the power necessary for the operation of the electronic components. To do so, the power source 21 comprises at least one rechargeable battery 22 and a charging port 23. The at least one rechargeable battery 22 enables the prolonged operation of the present invention, while the charging port 23 enables the recharging of the at least one rechargeable battery 22. The charging port 23 is externally integrated onto the first coupler 5 so that the charging port 23 is accessible to a power cable when not in use. Further, the at least one rechargeable battery 22 is electrically connected to the charging port 23, the controller 19, and the tachometer 20. This way, the user can recharge the at least one rechargeable battery 22 can always be fully charged so that the user can exercise for the desired period of time.

As can be seen in FIG. 1 through 5, to protect the crossbar 7 as well as to improve the grip of the user on the crossbar 7, the present invention may further comprise a grip cover 28. The grip cover 28 is designed to cover the outer surface of the crossbar 7 to help the user maintain a good grip on the crossbar 7. To do so, the grip cover 28 is positioned around the crossbar 7 to cover the outer surface of the crossbar 7. To prevent the grip cover 28 from coming loose, the grip cover 28 is also externally mounted onto the crossbar 7. This way, the user can safely and securely hold onto the crossbar 7 during the exercise, and the user can also remove the grip cover 28 for replacement or to use the present invention without the grip cover 28. In other embodiments, the crossbar 7 may be covered with a layer of grip material.

As can be seen in FIG. 10, in some embodiments, the present invention may facilitate the progress tracking of the health of the user when exercising with the present invention via a software application such as, but not limited to, a mobile application. To do so, the control system 18 may further comprise a transmitter 25 to send the signals from the controller 19 to a remote server or a mobile computing device such as, but not limited to, a smartphone. Accordingly, the transmitter 25 is mounted within the crossbar 7 to protect the transmitter 25 from the surroundings. Further, the transmitter 25 is electronically connected to the controller 19 to receive the signals to be transmitted to the remote server or the mobile computing device. The transmitter 25 is also electrically connected to the power source 21 to receive the current necessary for the operation of the transmitter 25. Thus, the RPM of the bearing assembly 10 as well as other parameters can be transmitted to the software application for easy tracking. Some of the other parameters can include the overall exercise time, heart rate, calorie burn, and other relevant health data. In other embodiments, other smart features can be equipped to the present invention.

Although the invention has been explained in relation to its preferred embodiment, it is to be understood that many other possible modifications and variations can be made without departing from the spirit and scope of the invention.

What is claimed is:

1. A handheld exercise device comprising:
an O-ring housing;
a crossbar;
a bearing assembly;
a control system;
the O-ring housing comprising a hollow ring body, a first coupler, and a second coupler;
the crossbar comprising a first crossbar end and a second crossbar end;
the control system comprising a controller, a tachometer, and a power source;
the first coupler being positioned opposite to the second coupler across the hollow ring body;
the first crossbar end being connected to the hollow ring body by the first coupler;
the second crossbar end being connected to the hollow ring body by the second coupler;
the controller and the power source being mounted within the crossbar;
the bearing assembly being slidably mounted within the hollow ring body;
the tachometer being electronically connected to the controller;
the tachometer and the controller being electrically connected to the power source; and
the tachometer being operatively coupled to the bearing assembly, wherein the tachometer is used to measure the number of revolutions the bearing assembly has completed along the hollow ring body in a period of time.

2. The handheld exercise device as claimed in claim 1, wherein the hollow ring body is made from a translucent plastic material.

3. The handheld exercise device as claimed in claim 1 comprising:
the bearing assembly comprising a plurality of bearings and a plurality of magnets;
the tachometer being a magnetic tachometer;
the plurality of magnets being interspersed among the plurality of bearings within the hollow ring body;
the tachometer being positioned adjacent to the first coupler; and
the tachometer being mounted within the hollow ring body.

4. The handheld exercise device as claimed in claim 3 comprising:
the bearing assembly further comprising a wire retainer;
the wire retainer comprising a first wire end and a second wire end;
the plurality of bearings comprising a first terminal bearing and a second terminal bearing;
the plurality of bearings and the plurality of magnets being distributed along the wire retainer;
the wire retainer traversing through each of the plurality of bearings and the plurality of magnets;
the first wire end being connected to the first terminal bearing; and
the second wire end being connected to the second terminal bearing.

5. The handheld exercise device as claimed in claim 3 comprising:
the control system further comprising a display;
the display being externally integrated onto the first coupler;
the display being electronically connected to the controller; and
the display being electrically connected to the power source.

6. The handheld exercise device as claimed in claim 3 comprising:
a plurality of magnetically activated lights;
the plurality of magnetically activated lights being radially distributed along the hollow ring body;
each of the plurality of magnetically activated lights being externally integrated onto the hollow ring body; and
the plurality of magnetically activated lights being electrically connected to the power source.

7. The handheld exercise device as claimed in claim 6 comprising:
a power switch;
the power switch being externally integrated onto the first coupler; and
the plurality of magnetically activated lights being electrically connected to the power source by the power switch.

8. The handheld exercise device as claimed in claim 1 comprising:
the power source comprising at least one rechargeable battery and a charging port;
the charging port being externally integrated onto the first coupler; and
the at least one rechargeable battery being electrically connected to the charging port, the controller, and the tachometer.

9. The handheld exercise device as claimed in claim 1 comprising:
a grip cover;
the grip cover being positioned around the crossbar; and
the grip cover being externally mounted onto the crossbar.

10. The handheld exercise device as claimed in claim 1 comprising:
the control system further comprising a transmitter;
the transmitter being mounted within the crossbar;
the transmitter being electronically connected to the controller; and
the transmitter being electrically connected to the power source.

11. A handheld exercise device comprising:
an O-ring housing;
a crossbar;
a bearing assembly;
a control system;
the O-ring housing comprising a hollow ring body, a first coupler, and a second coupler;
the crossbar comprising a first crossbar end and a second crossbar end;
the control system comprising a controller, a tachometer, and a power source;
the bearing assembly comprising a plurality of bearings and a plurality of magnets;
the tachometer being a magnetic tachometer;
the hollow ring body being made from a translucent plastic material;
the first coupler being positioned opposite to the second coupler across the hollow ring body;
the first crossbar end being connected to the hollow ring body by the first coupler;
the second crossbar end being connected to the hollow ring body by the second coupler;
the controller and the power source being mounted within the crossbar;
the bearing assembly being slidably mounted within the hollow ring body;
the plurality of magnets being interspersed among the plurality of bearings within the hollow ring body;
the tachometer being positioned adjacent to the first coupler;
the tachometer being mounted within the hollow ring body;
the tachometer being electronically connected to the controller;
the tachometer and the controller being electrically connected to the power source; and
the tachometer being operatively coupled to the bearing assembly, wherein the tachometer is used to measure the number of revolutions the bearing assembly has completed along the hollow ring body in a period of time.

12. The handheld exercise device as claimed in claim 11 comprising:
the bearing assembly further comprising a wire retainer;
the wire retainer comprising a first wire end and a second wire end;
the plurality of bearings comprising a first terminal bearing and a second terminal bearing;
the plurality of bearings and the plurality of magnets being distributed along the wire retainer;
the wire retainer traversing through each of the plurality of bearings and the plurality of magnets;
the first wire end being connected to the first terminal bearing; and
the second wire end being connected to the second terminal bearing.

13. The handheld exercise device as claimed in claim 11 comprising:
the control system further comprising a display and a transmitter;
the display being externally integrated onto the first coupler;
the display being electronically connected to the controller;
the display being electrically connected to the power source;
the transmitter being mounted within the crossbar;
the transmitter being electronically connected to the controller; and
the transmitter being electrically connected to the power source.

14. The handheld exercise device as claimed in claim 11 comprising:
a power switch;
a plurality of magnetically activated lights;
the plurality of magnetically activated lights being radially distributed along the hollow ring body;
each of the plurality of magnetically activated lights being externally integrated onto the hollow ring body;
the plurality of magnetically activated lights being electrically connected to the power source;
the power switch being externally integrated onto the first coupler; and
the plurality of magnetically activated lights being electrically connected to the power source by the power switch.

15. The handheld exercise device as claimed in claim 11 comprising:
the power source comprising at least one rechargeable battery and a charging port;
the charging port being externally integrated onto the first coupler; and
the at least one rechargeable battery being electrically connected to the charging port, the controller, and the tachometer.

16. The handheld exercise device as claimed in claim 11 comprising:
a grip cover;
the grip cover being positioned around the crossbar; and
the grip cover being externally mounted onto the crossbar.

17. A handheld exercise device comprising:
an O-ring housing;
a crossbar;
a bearing assembly;
a control system;
the O-ring housing comprising a hollow ring body, a first coupler, and a second coupler;
the crossbar comprising a first crossbar end and a second crossbar end;
the control system comprising a controller, a tachometer, and a power source;
the bearing assembly comprising a plurality of bearings, a plurality of magnets, and a wire retainer;
the wire retainer comprising a first wire end and a second wire end;
the plurality of bearings comprising a first terminal bearing and a second terminal bearing;
the tachometer being a magnetic tachometer;
the hollow ring body being made from a translucent plastic material;
the first coupler being positioned opposite to the second coupler across the hollow ring body;
the first crossbar end being connected to the hollow ring body by the first coupler;
the second crossbar end being connected to the hollow ring body by the second coupler;
the controller and the power source being mounted within the crossbar;
the bearing assembly being slidably mounted within the hollow ring body;
the plurality of magnets being interspersed among the plurality of bearings within the hollow ring body;
the plurality of bearings and the plurality of magnets being distributed along the wire retainer;
the wire retainer traversing through each of the plurality of bearings and the plurality of magnets;
the first wire end being connected to the first terminal bearing;
the second wire end being connected to the second terminal bearing;
the tachometer being positioned adjacent to the first coupler;
the tachometer being mounted within the hollow ring body;
the tachometer being electronically connected to the controller;
the tachometer and the controller being electrically connected to the power source; and
the tachometer being operatively coupled to the bearing assembly, wherein the tachometer is used to measure the number of revolutions the bearing assembly has completed along the hollow ring body in a period of time.

18. The handheld exercise device as claimed in claim 17 comprising:
the control system further comprising a display and a transmitter;
the display being externally integrated onto the first coupler;
the display being electronically connected to the controller;
the display being electrically connected to the power source;
the transmitter being mounted within the crossbar;
the transmitter being electronically connected to the controller; and
the transmitter being electrically connected to the power source.

19. The handheld exercise device as claimed in claim 17 comprising:
a grip cover;
a power switch;
a plurality of magnetically activated lights;
the plurality of magnetically activated lights being radially distributed along the hollow ring body;
each of the plurality of magnetically activated lights being externally integrated onto the hollow ring body;
the plurality of magnetically activated lights being electrically connected to the power source;
the power switch being externally integrated onto the first coupler;
the plurality of magnetically activated lights being electrically connected to the power source by the power switch;
the grip cover being positioned around the crossbar; and
the grip cover being externally mounted onto the crossbar.

20. The handheld exercise device as claimed in claim 17 comprising:
the power source comprising at least one rechargeable battery and a charging port;
the charging port being externally integrated onto the first coupler; and
the at least one rechargeable battery being electrically connected to the charging port, the controller, and the tachometer.
